(19) United States
(12) Patent Application Publication
(10) Pub. No.: US 2014/0263059 A1
(43) Pub. Date: Sep. 18, 2014

(54) PLASMA SEPARATION FROM BLOOD USING A FILTRATION DEVICE AND METHODS THEREOF

(71) Applicants: Lawrence J. Burg, Bedford, MA (US); Russel Rines, Bedford, MA (US); Aravind Srinivasan, Bedford, MA (US)

(72) Inventors: Lawrence J. Burg, Bedford, MA (US); Russel Rines, Bedford, MA (US); Aravind Srinivasan, Bedford, MA (US)

(21) Appl. No.: 14/202,447

(22) Filed: Mar. 10, 2014

Related U.S. Application Data
(60) Provisional application No. 61/783,696, filed on Mar. 14, 2013.

(51) Int. Cl. A61M 1/34; A61M 1/36; B01D 69/12

(52) U.S. Cl. CPC A61M 1/3496; B01D 69/12; A61M 1/3672
USPC 210/639; 210/321.6; 210/651; 156/280

(57) ABSTRACT

The invention is directed to a method and a device for separating plasma from whole blood. The method combines size exclusion filtration through a separation membrane and erythrocyte (RBC) agglutination.

### CROSS-REFERENCES TO RELATED APPLICATION

This application claims priority to and benefit of U.S. Provisional Application No. 61/783,696, filed Mar. 14, 2013, the entire contents of which are incorporated by reference herein for all purposes.

### BACKGROUND

Preparing clinical specimens for use in in vitro diagnostic devices is an essential step in achieving accurate and reliable results. The detection of intra-cellular components such as nucleic acids and proteins typically requires those cells to be lysed by chemical, mechanical or enzymatic means (or a combination thereof). The optimal method depends mainly on the specific cell that harbors the target antigen and spans all types of cells of animal, plant, fungal, bacterial and archae organisms. Specimen processing is also an important consideration for detecting non-cellular pathogens such as viruses and their components. Often, depending on the technology utilized in a diagnostic test, steps are required to further purify and/or concentrate the cells or antigen from the specimen or crude lysate to enable effective detection. Another important consideration is the type of biological specimen itself as methods vary considerably for tissue samples, blood, sputum, feces, urine, saliva, lavage samples, plural fluid, etc. In all cases, the essential goal of specimen processing is to decrease the complexity of the test sample by removing potential interfering substances, preserving the target antigen's structure and presenting the antigen in a form that can be readily detected. Concentrating the antigen to improve analytical sensitivity may also be an important objective of specimen processing.

In many cases, the antigen to be detected exists as an extracellular entity which obviates the need for cellular lysis. However, such antigens may still require specimen processing steps to achieve adequate test performance. An important category of such antigens are those present in the plasma constituent of blood. A variety of human diseases cause cellular damage leading to the appearance or elevation of antigens in the blood. For example, heart attacks cause damage to cardiac myocytes which release cardiac troponins into the blood stream. Similarly, heart failure involving the left ventricle leads to the release of BNP and NT-proBNP into the blood. Thus the presence and amount of these cardiac markers (antigens) in plasma have high diagnostic value for identifying clinical disease and are important in the overall management of these patients.

Blood, however, is a complex biological specimen which is about 5 times more viscous than water. Blood contains many sub-components, including plasma proteins (e.g. albumin, globulins, fibrinogen), enzymes, metabolites, inorganic substances (e.g. calcium, sodium, magnesium, potassium) carbohydrates, lipids, vitamins, hormones, dissolved gases, leukocytes (i.e. white cells), erythrocytes (i.e. red blood cells or RBC), thrombocytes (i.e. platelets) and other components (e.g. creatine, amino acids, choline, histamine, bilirubin). In particular, erythrocytes comprise about 43% of the volume of blood, although this varies with age, gender and disease state. This equates to about 5 million RBC per microliter of blood whereas there are about 7,000 leukocytes in the same volume. Furthermore, once removed from the body, blood will quickly develop clots which further complicate specimen handling and antigen detection.

The clotting problem has been solved simply by adding an anticoagulant to the tube used to collect the blood specimen. Anticoagulants prevent clotting and are routinely used. Examples of anticoagulants include heparin, EDTA and sodium citrate. However, the large number of RBC and leukocytes in blood and the substantial volume they occupy often prove problematic for diagnostic instrumentation systems and assays intended to detect and/or quantify antigens, for example the cardiac antigens above, in plasma. Many diagnostic systems simply are not technically equipped to handle anticoagulated blood specimens. Instead, they rely on the user to process blood specimens by separating the plasma from the cells and then adding the plasma sample to the diagnostic system. Fortunately, this is relatively straightforward as low speed centrifugation of blood collection tubes is all that is required to pellet the cells, leaving the plasma as a separate layer on top of the cells. However, this step does require a centrifuge and roughly 15 minutes to perform and can present additional biohazardous exposure. In some settings, such as emergency departments, centrifugation of blood is simply not acceptable with respect to workflow, time required, exposure concerns and equipment (centrifuges are not typically available).

Thus many diagnostic device manufacturers have tried to solve the problem of automatically processing blood specimens on their systems. Some manufacturers have gone so far as to incorporate centrifugal separation mechanics into their instruments. Not only does the centrifugal mechanics add considerable complexity to the system, it also adds substantial size to the instrument and may require an additional expensive disposable plastic accessory. Some manufacturers have developed lateral flow devices which incorporate layers of absorbent and membrane materials that serve to retard blood cells and wick the plasma along a substrate layer (typically nitrocellulose) to a detection zone. These devices don't achieve true plasma separation but rather take advantage of the size of cells to slow their progress in lateral flow relative to the plasma. Regardless, these devices, although easy to use and produce rapid results, are generally not very quantitative or sensitive and therefore lack clinical utility in many situations.

Others have circumvented the entire problem by employing specialized techniques and features to allow plasma antigens to be detected in anticoagulated blood without the need to physically separate the plasma from blood cells. Such methods show promise but few have made it to market presumably due to the complexity, cost and reliability of implementing the technology or to the sacrifice incurred in assay performance (e.g. loss of sensitivity, accuracy, precision or dynamic range).

Thus, there exists the need for a simple, reliable, low cost, easy to use means to prepare plasma from blood which preserves antigen conformation, retains antigen concentration and yields sufficient volume for diagnostic purposes.

### SUMMARY OF THE INVENTION

Generally, the invention described herein is directed to a separation device and method that rapidly separates the non-cellular fluid portion of blood, for example, plasma, from anti-coagulated whole blood without the loss of antigens, particularly antigens that exist in the fluid portion of blood as an extracellular entity. The invention described herein has many advantages over prior art plasma separation devices and methods, for example and without limitation: ease of use, scalability, automation, ease of fabrication, stability and reliability, high recovery of plasma antigens, high plasma yield, low cost, and applicability to high hematocrit blood specimens, various anti-coagulants, and flexibility for use in many applications and configurations.

In one aspect, the invention is directed to a plasma separation device comprising a housing and a filtration (separation) membrane. In one embodiment of the invention, the housing has a blood introducing port and a plasma outflow channel. The filtration membrane has a blood contact side and an opposite side, and in one embodiment, comprises polysulfone-PVP or polysulfone. The filtration membrane features a plurality of pores, has a thickness of about 200 to about 400 μm, for example, 400 μm, is coated with a hemagglutination agent, and, in one embodiment, further comprises a coating of bovine serum albumin. The filtration membrane is positioned between the blood introducing port where a whole blood sample is introduced, and the plasma outflow channel of the housing where plasma, after cells are removed while blood flows through the filtration membrane, is collected.

In one embodiment of the invention, the hemagglutination agent is selected from the group consisting of lectins, polyvinyl sulfate, polymers and heparins. Agglutinins and such lectins may be selected from the group consisting of AAL, PSA, STL, ConA, SNA, LCA, PHA-E, WGS, s-WGA, and DSL, and combinations thereof.

In one embodiment of the plasma separation device according to the invention, the plurality of pores of the filtration membrane comprise internal surfaces that are coated with the hemagglutination agent. The plurality of pores range in size from about 30 to 350 μm on the blood contact side of the filtration membrane and from about 0.8 to 2 μm on the opposite side of the filtration membrane, for example, about 1.5 μm. In a particular embodiment of the plasma separation device, the average diameter of a pore of the filtration membrane on the blood contact side of the filtration membrane is greater than the average diameter of the pore on the opposite side of the filtration membrane.

In another aspect, the invention is directed to a method for separating the non-cellular fluid portion of blood, for example, plasma, from whole blood. The steps of the method comprise, contacting a whole blood sample with a hemagglutination agent, filtering the blood sample through a filtration membrane comprising pores in the size range of about 0.8 μm to about 350 μm, the membrane thickness about 200 to about 400 μm, and collecting the non-cellular fluid portion of the whole blood sample from the filtered whole blood sample at the plasma outflow channel of the separation device. In one embodiment of the invention, the whole blood sample is contacted with the hemagglutination agent which is coated on the filtration membrane, or, alternatively, by the direct addition of the hemagglutination agent to the blood sample. In a particular embodiment of the method of the invention, an anti-coagulant is added to the whole blood sample.

In one embodiment, the whole blood sample is filtered under pressure applied to the pores of the filtration membrane from the blood contact side of the membrane. Alternatively, in yet another embodiment, the whole blood sample is filtered in the presence of a vacuum applied on the opposite side of the membrane to which the blood sample is added, or, alternatively, is filtered under capillary forces of the pores of the membrane.

According to one embodiment of the method of the invention for separating the non-cellular portions of blood from whole blood, antigens recovered in the non-cellular fluid portion of whole blood filtered through the separation membrane described above is greater than 80% compared to antigen recovered in plasma prepared by centrifugation of whole blood to remove cellular elements. In one embodiment, the recovered antigen is a cardiac marker, for example, troponin, NT-pro-BNP, pro-BNP, BNP, or other naturietic peptides.

In yet another aspect, the invention is directed to a method for manufacturing a plasma separation device for separating the non-cellular portion of blood, plasma for example, from whole blood. The method of manufacture comprises the steps of bonding a filtration membrane having a plurality of pores, a blood contact side and an opposite side, to a housing. The housing may be a component of a microfluidic device. The filtration membrane is coated with a hemagglutination agent by contacting the filtration membrane with the hemagglutination agent. A filtration membrane may be coated with a hemagglutination agent by, for example, heat drying, vacuum drying, dipping, or rolling the hemagglutination agent with the filtration membrane to apply the hemagglutination agent to the filtration membrane.

In one embodiment, the hemagglutination agent may be selected from the group of lectins and agglutinins consisting of AAL, PSA, STL, ConA, SNA, LCA, PHA-E, WGS, s-WGA, and DSL, and combinations thereof.

In one embodiment according to the invention, the filtration membrane is coated with a hemagglutination agent by coating the internal surfaces of the pores of the filtration membrane under capillary forces applied to the pores of the filtration membrane.

In yet another embodiment according to the invention, the filtration membrane is coated with a hemagglutination agent by coating the internal surfaces of the pores of the filtration membrane in the presence of positive pressure applied to the pores from the blood contact side of the filtration membrane.

In another embodiment according to the invention. the filtration membrane is coated with a hemagglutination agent by coating the internal surfaces of the pores of the filtration membrane in the presence of a vacuum applied to the pores on the opposite of the filtration membrane.

### BRIEF DESCRIPTION OF THE DRAWINGS

FIG. 1 schematically illustrates a transverse cross-section of an exemplary plasma separation device and a pore size indicator according to one embodiment of the invention.

FIG. 2 schematically illustrates a transverse cross-section of the plasma separation device illustrated in FIG. 1 included as a component of a microfluidic device.

FIG. 3 illustrates the plasma separation device illustrated in FIG. 1 with a hemagglutination agent coated membrane.

FIG. 4 is a graph illustrating the plasma yield from untreated and STL-treated separation membrane.

FIG. 5 illustrates a correlation plot of troponin in plasma prepared by filtration through STL-treated separation membrane versus conventional centrifugation.

### DESCRIPTION OF THE INVENTION

In one aspect, referring now to FIG. 1, the invention described herein is directed to a plasma separation device 100 having a housing 90, a blood introducing entry port 92, a plasma outflow channel 94, and a filtration membrane 80 positioned between the blood introducing entry port 92 and the plasma outflow channel. The separation device 100, according to the invention, is useful for separating non-cellular components of blood, such as plasma 102, from cells 96 of blood, e.g, red blood cells and white blood cells. In one embodiment, the separation device 100 is joined to or is a component of a microfluidic device 10 for analyzing analytes in a patient blood sample, as shown, for example, in FIG. 2.

Referring again to FIG. 1, by positioning the plasma filtration (also termed separation) membrane 80 between the blood introducing entry port 92 and the plasma outflow channel 94 it is meant that whole blood that is introduced through the blood introducing entry port 92 flows from the entry port 92 through the separation membrane 80. In the separation membrane 80, plasma is separated from the cellular components of blood, and the plasma 102, now substantially free of blood cells 96, is released from the filtration membrane 80 into the plasma outflow channel 94 from which the plasma 102 is ultimately collected.

The filtration or separation membrane 80, as it is also called, is made of suitable filtration membrane materials including but not limited to polysulfone, polysulfone-PVP, glass fiber, and cellulose acetate to name a few of such materials. The filtration membrane 80 is typically planar and may have a thickness in the range of about 200-800 μm, 300-600 μm, 350-450 μm, preferably, about 400 μm. The filtration membrane 80 has a blood contact side 82 that comes in contact with a blood sample introduced via the blood entry introducing port 92, and an opposite side 86. The filtration membrane 80 includes a plurality of pores 84 extending through the filtration membrane 80 from the surface at the blood sample contact side 82 through the mesh or body 83 of the filtration membrane 80, and finally through the surface at the opposite side 86 of the filtration membrane 80. The term “pore” as used herein pertains to a tortuous path in a lattice-like mesh 83 of the filtration membrane 80. A pore 84 may extend from one surface of the filtration membrane 80 to the opposite surface via a path that may be contorted and indirect as compared to a path that takes a route that extends perpendicular to the blood contact surface or the opposite surface of the filtration membrane. Typically, the diameter of the average pore is in the range of about 30 μm to about 350 μm on the blood contact side 82 and diminishes to about 0.8 μm to about 2.0 μm on the opposite side 86 of the filtration membrane 80.

Referring to FIG. 3, in one embodiment, the separation membrane 80 is coated with at least one hemagglutination agent 88, for example but not limited to lectins or agglutinins such as AAL, PSA, STL, ConA, SNA, LCA, PHA-E, WGS, s-WGA, and DSL, as denoted in Table II below, and/or polyvinyl sulfate, polymers, heparin, and combinations thereof. In one embodiment of the invention, all membrane surfaces include not only the surfaces of the outside of the separation membrane 80 such as blood contact surface 82 and opposite side surface 86, but also the internal surfaces 85 of the pores 84 are similarly coated with the hemagglutination agent 88.

Typically, the filtration membrane 80 has pores 84 on the blood contact side 82 of the membrane that are greater in average diameter than the average diameter of pores 84 on the opposite side 86 of the membrane.

In one embodiment of the invention, the filtration membrane 80 further comprises a coating of bovine serum albumin.

In another aspect, the invention is directed to a method for separating plasma from a whole blood sample. The whole blood sample is introduced into the plasma separation device discussed above through the blood introducing entry porl and is contacted with a hemagglutination agent. In one embodiment, the hemagglutination agent is located on the surfaces of the separation membrane, including the surfaces of the pores. The blood percolates through the pores of the filtration membrane by, for example, capillary action, a vacuum (negative pressure), or positive pressure applied to the membrane. The filtered blood exits the opposite side of the separation membrane into the plasma flow channel from which the plasma is collected.

In another aspect, the invention is directed to a method for manufacturing the plasma separation device described above. A separation membrane, as described above, is bonded to a housing, for example a plastic housing, by methods known to the skilled person such as but not limited to thermal bonding, and/or application of an adhesive. The plastic housing may be joined to a microfluidic device for analyzing analytes in a patient blood sample or may be a component of a microfluidic device. The separation membrane is coated with a hemagglutination agent as discussed above, preferably by contacting the hemagglutination agent with the blood contact side of an asymmetrical membrane that has pores with a larger average diameter than the pores on the opposite side of the asymmetrical membrane. Coating the separation membrane with a hemagglutination agent is accomplished by methods known by the skilled artisan, for example but not limited to, heat drying, vacuum drying, applying an adhesive, dipping, and rolling the filtration membrane to apply the hemagglutination agent to the membrane. Capillary forces, the application of positive pressure, or the application of a vacuum to the pores of the separation membrane may be used to draw the hemagglutination agent into the pores of the separation membrane.

In one aspect, the invention is directed to a method to separate plasma from anticoagulated blood. The fundamental principle of the method of the invention is size exclusion filtration. The invention described herein resolves numerous problems of prior art plasma separation devices and methods, including undesirable complications such as RBC leakage, poor blood flow rates through the plasma separation device, separation membrane clogging, antigen loss, low plasma volume yield, poor quality plasma, for example, hemolyzed plasma, and inconsistencies between one lot compared to another lot that characterize prior art plasma separation devices.

While developing the method and device for plasma separation disclosed herein, various experimental treatments to the membrane (e.g. with casein, sucrose, Tween at elevated pH) were investigated. These treatments improved antigen recovery but were less desirable with respect to slow blood flow, sample hemolysis, and in some batches of material, substantial RBC leakage. Many other components were evaluated as membrane treatments (see Table I below). What is shown is merely illustrative of what was investigated since it does not include the range of concentrations that were tested, the range of polymer molecular weights that were tested and, importantly, the various combinations of these elements that were tested.

Results of these studies included no effects, deleterious effects and improving performance in one area but decreasing performance in another (e.g. increased flow rate or lack of hemolysis but with the loss of antigen). A desirable treatment solution that resolved the poor flow rates or hemolysis while simultaneously preserving antigen did not emerge from these initial exploratory studies.

TABLE I
Membrane Treatment Chemistries Evaluated

| Component Type | Specific Examples |
|---|---|
| Polymers | PVA, PVP, PEO, Dextran |
| Protein | BSA, Casein, Peptides (created by proteolysis) |
| Surfactants | Tween-20, Tween-40, NP-40, Triton X-100, FS8050, |
| | FS8150 and FS8250 (fluorosurfactants) |
| Sugar | Sucrose, Glucose |
| Small Molecule | NaCl, Glycine, Lysine, Valine, Leucine, Isoleucine, |
| | Phenylalanine, Poly-L-Lysine |
| Buffers | Phosphate, Tris, MES, varying pH |

Referring generally to FIGS. 1-3, the invention described herein is directed to a method and a device for separating plasma from whole blood. The method combines size exclusion filtration through a separation (filtration) membrane and erythrocyte (RBC) agglutination. Aggregating RBCs dramatically improves filtration rates by reducing the number of individual RBCs that otherwise infiltrate the pore structure of the separation membrane through which the blood sample is filtered and clog the porous openings of the membrane.

The separation membrane according to the invention may be made of polysulfone, polysulfone-PVP, cellulose acetate, glass fiber, and a variety of polymers to name a few of the possible membrane materials.

In one embodiment of the invention, the average pore size of an asymmetric (asymmetric meaning the average diameter of the pores on one side of the membrane differs from the average diameter of the pores on the other side of the membrane) polysulfone membrane is about 0.8 μm to about 2.0 μm, preferably 1.3 μm. We have found that 1.3 μm is effective in preventing RBCs from leaking through the separation membrane. However, the greater restriction caused by the smaller pores also substantially reduces flow rates (0.04 microL/sec to 0.01 microL/sec), particularly at higher blood hematocrits (corresponding to the volume of packed cells in blood) and with chemical membrane treatments, such as casein, sucrose, Tween®, polymers (see Table I above). This flow restriction is largely overcome by preventing RBCs from clogging the pores by creating large RBC aggregates that can not penetrate deep into the pores of the separation membrane structure. In this regard, increasing the filtration capacity of the separation membrane by increasing the separation membrane thickness to a range between about 200-800 μm, 300-600 μm, 350-450 μm, preferably, about 400 μm has also proven to be important. Thus, a polysulfone-PVP separation membrane of about 400 μm thick, with average pore structure ranging from about 200 μm on one side to about 1 μm on the other side (hereafter referred to as ILM membrane) and with a RBC agglutination mechanism, described below, solves the major problems associated with efficient plasma separation from anticoagulated blood. A desired flow rate would be about 0.15 microL/sec to about 1.0 microL/sec. However, much larger flow rates are possible with larger membrane surface areas. The plasma separation device and method of plasma separation described herein permits rapid flow rates, high antigen recovery and, high yield of high quality plasma.

In one embodiment of the method of the invention, the agglutination of RBCs in an anti-coagulated whole blood specimen is achieved by the direct addition of hemagglutination components to the blood prior to contacting the blood sample with the membrane filtration system. For example, the addition of large amounts of heparin or polymers invokes RBC aggregation thought to be induced by a depletion mechanism at the boundary layer between the cell surface and the co-solute. Addition of 34 mg/mL heparin to a 48% hematocrit blood specimen which was then filtered through an ILM untreated separation membrane (i.e., not coated with a hemagglutination agent) led to a plasma separation time of only 18 seconds compared to 3.5 minutes without heparin. Similar results were achieved by the addition of a synthetic polymer (16 mg/mL polyvinyl sulfate; molecular weight of 170,000 g/mol) to blood prior to filtering through the separation membrane. Yet further similar results were achieved with a variety of lectins and agglutinins (AAL, PSA, STL, ConA, SNA, LCA, PHA-E, WGA, s-WGA, DSL, see table of lectins and agglutinins attached below at Table II) added to blood prior to filtration which caused hemagglutination by the specific interaction and crosslinking of carbohydrates of cell surface glycoproteins. Due to the specific agglutination mechanism, much smaller amounts (about 0.2 mg/mL) of lectins were effective for achieving these rapid flow rates and plasma separation compared to the other potential hemagglutination agents mentioned above. In all cases, aggregation of RBC was confirmed by microscopic visualization.

TABLE II
Table of Lectins

| Abbreviation | Full Name |
|---|---|
| AAL | Aleuria aurantia lectin |
| PSA | Pisum sativum lectin |
| STL | Solanum tuberosum lectin |
| ConA | Concanavalin A |
| SNA | Sambucus nigra Agglutinin |
| LCA | Lens Culinaris Agglutinin |
| PHA-E | Phaseous Vulgaris Erythroagglutinin |
| WGA | Wheat Germ Agglutinin |
| s-WGA | Succinylated Wheat Germ Agglutinin |
| DSL | Datura Stramonium lectin |

However, direct addition of a RBC agglutination agent to blood is not easy to automate in a diagnostic instrument. Furthermore, such a step is not practical in some clinical settings such as the emergency room due to the time and extra steps, the storage of the additive, the need for dispensing equipment, the requirement of careful and accurate delivery, additional exposure to biohazards, and adulterating the specimen which limits its use for other diagnostic tests.

The practical problems mentioned above were solved by exposing the side of the separation membrane (blood contact side of the membrane described above in reference to FIG. 3) that will come in contact with the blood sample (i.e., the larger pore side compared to the pore side on the opposite side of the separation membrane) with a hemagglutination agent in sufficient volume for a sufficient length of time to ensure that the hemagglutination agent penetrates throughout the interstices of the separation membrane from the blood contact side of the separation membrane to the opposite side of the separation membrane bringing the hemagglutination agent in contact with the internal surfaces of the pores. The challenges in selecting an appropriate hemagglutination agent is that the hemagglutination agent must be stable on the membrane surfaces after drying, even after prolonged storage. Also, the hemagglutination agent must be readily solubilized by the blood specimen and must rapidly regain its activity once solubilized. Once soluble and active, the hemagglutination agent must rapidly aggregate red blood cells before the red blood cells clog the separation membrane pores since the blood specimen is rapidly pulled by capillary forces from the blood contact side of the separation membrane into the membrane. The hemagglutination agent coated on the surfaces of the separation membrane aggregates only enough of the RBCs in the sample necessary to inhibit clogging of the membrane. In other words, it is not necessary for the hemagglutination agent to diffuse throughout the entire blood sample volume. RBC agglutination is a dynamic process involving those cells moving into the separation membrane pores.

As opposed to their direct addition to blood, heparin and polyvinyl sulfate proved largely ineffective for treating membranes as only slight improvements were seen for plasma separation times. This is likely due to insufficient amounts of these agents after drying onto the separation membrane and subsequent solubilization since the non-specific aggregation of RBC by these agents requires high concentrations.

In contrast, lectin treatment of ILM membranes proved to be highly effective for plasma separation and is a specific example of the implementation of this invention for rapidly collecting plasma from blood by filtration without loss of plasma antigens. Minor hemolysis of the specimen, should it occur, could be mitigated by the co-treatment of the ILM membrane with lectin and bovine serum albumin (BSA).

### Examples

ILM untreated separation membrane discs (13 mm diameter) were heat-bonded to polystyrene housings designed for a microfluidic cartridge. Ten microliters (10 uL) of a solution containing 0.2 mg/mL Solanum tuberosum lectin (STL) in 10 mM HEPES pH 7.5, 0.1 mM CaCl₂ was contacted with the large pore side (side that will come in contact with a blood sample) of the separation membrane. The separation membrane was then dried in a vacuum oven at 34° C. for 30 minutes. In an alternate embodiment, not used in this example, larger pieces of the separation membrane could be treated with the hemagglutination agent first, an appropriate piece of the treated separation membrane would be cut out and then bonded to the housing.

We conducted a study in which 12 separate troponin-spiked blood samples (average 41% hematocrit, range 36-45%) were added to the STL-treated side (large pore, i.e., blood contact side) of ILM membranes described above and the plasma was separated from the blood sample by filtration through the treated separation membrane. The resulting plasma was harvested by vacuum into a channel within the microfluidic cartridge. About 5.2 uL of plasma was pulled into the device before the vacuum was turned off. The average plasma separation time (defined as time from vacuum on until vacuum off) was only 17 sec with a range of 11 to 26 sec. Thus, very rapid flow rates of blood through the separation membrane were achieved by the lectin treatment resulting in very rapid separation of plasma from whole blood. Furthermore, the average troponin (TnI) recovery was 94% (range 79% to 104%). The data is summarized in Table III below.

TABLE III
Plasma Separation Time and Antigen Recovery using
STL-Treated ILM Membrane

| | | Tnl | | |
|---|---|---|---|---|
| | | Recovery | Blood | |
| | | (WB/ | Separation | |
| Donor | Hct | Plasma) | (sec) | |
| #309 | 36% | 97% | 12 | |
| #223 | 44% | 97% | 20 | |
| #141 | 40% | 97% | 11 | |
| #73 | 41% | 98% | 18 | |
| #356 | 44% | 79% | 21 | |
| #316 | 45% | 92% | 26 | |
| #139 | 39% | 99% | 13 | |
| #102 | 41% | 94% | 14 | |
| #33 | 43% | 86% | 17 | |
| #315 | 43% | 91% | 19 | |
| #235 | 39% | 95% | 14 | |
| #118 | 40% | 104% | 16 | |
| Avg | 41% | 94% | 17 | sec |
| Range | 36%-45% | 79%-104% | 11-26 | sec |

It should be noted that the 5.2 uL plasma yield is all the volume that is required in the microfluidic cartridge system to produce a diagnostic result and is not to be construed as the maximum yield of plasma through a STL-treated ILM membrane according to the invention.

In order to determine maximum yield, another study was conducted. Ninety (90) uL of blood was added to STL-treated or untreated ILM separation membrane (bonded to housings) and placed in direct contact with a pre-weighed stack of 13 mm diameter absorbent discs. After 3 minutes, the absorbent discs were weighed and the difference in mass used as a measure of the maximum volume of separated plasma. This experiment was conducted with 4 blood specimens with hematocrit ranging from 39% to 50%. As expected, with both the STL-treated and untreated separation membrane, the plasma yield declined with increasing hematocrit due to slower filtration rates and/or membrane clogging induced by more RBCs. In all cases, the plasma yield was greater with the STL-treated ILM membrane compared to the non-treated ILM membrane, averaging 42% more volume (range 32% to 55%) than the untreated membrane as shown graphically in FIG. 4 below. The STL-treated membrane yielded about 11.5 uL of plasma from the 50% hematocrit specimen. The percent yield can also be estimated. In 90 uL of a 50% hematocrit blood specimen, the maximum available plasma is 45 uL. The interstitial dead space of the ILM membrane (given the diameter of the disc and its thickness) has been measured to be about 35 uL. Material in this space will not be able to be harvested due to the extremely high capillary forces. If half of this space is occupied by plasma—the other half by blood cells—then the maximum plasma that could be obtained is 45 uL−(35 uL/2)=27.5 uL. The percent yield was therefore 11.5/27.5=42%.

STL-treated ILM membranes were further qualified in a study using prospectively collected blood specimens from patients with chest pain or other symptoms in which a cardiac troponin assay was requested. Thirty-eight patients were evaluated in this study. An aliquot from each patient's blood specimen was centrifuged to prepare plasma by conventional means. The blood and plasma specimens were then processed on the microfluidic cartridge system using STL-treated ILM membrane. The average recovery of troponin (determined by dividing the amount of troponin signal prepared by filtration of the blood sample through the lectin-treated membrane by the troponin signal in plasma prepared by centrifugation of the blood sample) was 95% (range 69% to 120%). Furthermore, correlation plots of the troponin concentrations in blood versus plasma showed excellent agreement with regression curves of y=1.002x (linear fit; r̂2=0.9924) or y=0.933x̂1.003 (power fit; r̂2=0.9969). The power fit results on a log-log plot are shown below in FIG. 5.

The plasma separation method according to the invention easily and rapidly separates plasma from anticoagulated whole blood without the loss of plasma antigens. A key feature of one embodiment of the invention is a polysulfone-PVP membrane with an asymmetric pore structure that is treated with an agent that induces RBC aggregation.

In a particular embodiment of the plasma filtration device of the invention, the membrane filter has small pores with diameters of about 0.8 to about 2.0 um. Lectins were shown to be effective agents to treat membranes and promote RBC aggregation and rapid filtration rates. The plasma membrane has, for example, 2 ug of Solanum tuberosum lectin dried onto the membrane. One skilled in the art will appreciate that this invention is not restricted to lectins and agglutinins disclosed herein as other agents which cause RBC aggregation may also be used, for example, heparin, polymers, and other agents, for example, as shown in Table II.

Also, the invention described herein is not restricted to the concentrations, method of application or drying conditions used in the illustrated examples. Furthermore, this invention may be applied to membranes made from materials other than polysulfone-PVP, and is not restricted to an asymmetric pore structure in which the average diameter of the pores on the side of the membrane that comes in contact with blood is larger than the average diameter of the pores on the opposite side of the membrane. Although the example described herein used a STL-treated membrane bonded to a housing which was attached to a microfluidic cartridge and the separated plasma was harvested into micro-channels of the microfluidic cartridge using vacuum, there are many ways to apply this invention to other device designs to yield separated plasma. For example, larger membrane areas or other membrane configurations (e.g. tangential flow arrangements) may be used and may increase the surface area and yield even more plasma volume.

A major advantage of the plasma separation method and device according to the invention described herein is the ease of use. In one embodiment of the invention, the membrane treatment method simply involves contacting a hemagglutination agent, for example, a solution of STL, to the membrane followed by a step such as drying to join the agent to the surfaces of the membrane.

The membrane preparation can readily be scaled and automated, for example by using dip tanks and rolls of membrane followed by rapid hot air knife or tunnel dryers. When whole blood is applied to the large pore side of STL-treated membrane, plasma separation begins immediately and without external forces or the need for any device or instrument. This occurs solely due to the capillary forces created within the pore structure of the membrane. Since the pores are smaller on the side of the membrane opposite to the side of the membrane which is in contact with the introduced blood sample, the capillary forces increase in the direction of the smaller pores and the plasma flows in that direction. Separated plasma accumulates automatically on the surface of the small pore side (opposite side) and can be harvested away from that surface by many means known to those skilled in the art, including those described herein, for example, vacuum or wicking with absorbent materials.

The invention therefore has many advantages for preparing plasma from blood for diagnostic applications. It is simple to fabricate. It is low cost. It is easy to use. It is reliable and stable. It permits very rapid plasma separation. It yields high recovery of plasma antigens. It yields high plasma recovery as a percentage of applied blood volume. It works with high hematocrit blood specimens and different anticoagulants. It is flexible to be used in many applications and configurations.

What is claimed is:

1. A plasma separation device, comprising:
a housing comprising a blood introducing port and a plasma outflow channel; and
a filtration membrane having a blood contact side and an opposite side, said filtration membrane comprising a plurality of pores and comprising a thickness of about 200 μm to about 400 μm, and wherein said filtration membrane is coated with a hemagglutination agent.

2. The plasma separation device of claim 1 wherein said filtration membrane is positioned between said blood introducing port and said plasma outflow channel.

3. The plasma separation device of claim 1 wherein said plurality of pores comprise internal surfaces coated with said hemagglutination agent.

4. The plasma separation device of claim 1 wherein said plurality of pores comprise a range from about 30 μm to 350 μm on the blood contact side of the membrane and about 0.8 μm to 2 μm on the opposite side of the membrane.

5. The plasma separation device of claim 1 wherein said plurality of pores on the filtration membrane comprise pores on the blood contact side of the membrane and pores on the opposite side of the membrane wherein an average diameter of the pores on the blood contact side are greater than the average diameter of the pores on the opposite side of the membrane.

6. The plasma separation device of claim 1 wherein said pores are in a diameter range of about 1.5 μm.

7. The plasma separation device of claim 1 wherein said hemagglutination agent is selected from the group consisting of lectins, polyvinyl sulfate, polymers, heparin and combinations thereof.

8. The plasma separation device of claim 1 wherein said filtration membrane further comprises a coating of bovine serum albumin.

9. The plasma separation device of claim 1 wherein said hemagglutination agent is selected from the group consisting of lectins and agglutinins consisting of AAL, PSA, STL, ConA, SNA, LCA, PHA-E, WGS, s-WGA, and DSL, and combinations thereof.

10. The plasma separation device of claim 1 wherein said thickness of said filtration membrane is about 400 μm.

11. The plasma separation device of claim 1 wherein said filtration membrane is selected from the group consisting of a polysulfone-PVP and a polysulfone.

12. A method for separating a non-cellular fluid portion of blood from whole blood comprising the steps of:
(a) contacting the whole blood sample with a hemagglutination agent;
(b) filtering said whole blood sample through a filtration membrane comprising pores comprising diameter a range of about 0.8 μm to about 350 μm and a thickness comprising a range of about 200 μm to about 400 μm; and,
(c) collecting said non-cellular fluid portion from the filtered whole blood sample.

13. The method of claim 12 wherein step (a) comprises contacting said whole blood sample with the hemagglutination agent while said hemagglutination agent is coated on said filtration membrane.

14. The method of claim 12 wherein said filtering occurs under capillary forces in the pores of the membrane.

15. The method of claim 12 wherein said filtering occurs in the presence of a vacuum applied on the side of the membrane opposite to which the whole blood sample is added.

16. The method of claim 12 wherein said filtering occurs under a pressure applied to the pores of the membrane from the blood contact side of the membrane.

17. A method for manufacturing a plasma separation device comprising:
bonding a filtration membrane to a plastic housing, said filtration membrane comprising a plurality of pores, a blood contact side and an opposite side; and
coating the filtration membrane with a hemagglutination agent by contacting the filtration membrane with the hemagglutination agent.

18. The method of claim 17 wherein said hemagglutination agent is selected from the group of lectins and agglutinins consisting of AAL, PSA, STL, ConA, SNA, LCA, PHA-E, WGS, s-WGA, DSL, and combinations thereof.

19. The method of claim 17 wherein the coating a filtration membrane comprises a step selected from the group consisting of heat drying, vacuum drying, dipping, and combinations thereof.

20. The method of claim 17 wherein said coating of the separation membrane with the hemagglutination agent comprises coating surfaces of the pores under capillary forces applied to the pores of the membrane.

21. The method of claim 17 wherein said coating of the membrane with the hemagglutination agent comprises coating the pores in the presence of a vacuum applied to the pores on the opposite side of the membrane.

22. The method of claim 17 wherein coating the membrane with the hemagglutination agent comprises coating the pores in the presence of a positive pressure applied to the pores from the blood contact side of the membrane.

23. The method of claim 12 further comprising adding an anticoagulant to the whole blood sample.

24. The method of claim 12 wherein said non-cellular fluid portion obtained from the filtered whole blood comprises a plasma.

25. The method of claim 12 wherein antigen recovery in the non-cellular fluid portion is greater than 80% compared to antigen recovery in a non-cellular fluid portion obtained by centrifugation to remove cellular elements.

26. The method of claim 25 wherein said antigen is a cardiac marker.

27. The method of claim 26 wherein said cardiac marker comprises a naturietic peptide.

28. The method of claim 1 further comprising rolling said filtration membrane to apply said hemagglutination agent to said membrane.

29. The method of claim 27 wherein the naturietic peptide is selected from the group consisting of troponin, NT-pro-BNP, pro-BNP, BNP, and combinations thereof.
